(19) United States
(12) Patent Application Publication
(10) Pub. No.: US 2025/0098960 A1
(43) Pub. Date: Mar. 27, 2025

(54) FEATURE SUBSCRIPTIONS FOR MEDICAL DEVICE SYSTEM FEATURE SETS

(71) Applicant: Medtronic, Inc., Minneapolis, MN (US)

(72) Inventors: Grant A. Neitzell, Loretto, MN (US); Abhijit P. Jejurkar, Plymouth, MN (US); Leonardo Rapallini, Edina, MN (US); Paul G. Krause, Mahtomedi, MN (US); Charles A. Sarbib-Brown, New Brighton, MN (US); Shuang Liang, Saint Paul, MN (US)

(21) Appl. No.: 18/832,237

(22) PCT Filed: Feb. 9, 2023
(86) PCT No.: PCT/US2023/062314

§ 371 Date: Jul. 23, 2024

Related U.S. Application Data
(60) Provisional application No. 63/267,825, filed on Feb. 10, 2022.

(51) Int. Cl. A61B 5/00; A61B 5/346; G06Q 30/04; G16H 40/20; G16H 40/67; G16H 50/30

(52) U.S. Cl. CPC A61B 5/0031; A61B 5/0006; A61B 5/0022; A61B 5/346; A61B 5/686; G06Q 30/04; G16H 40/20; G16H 40/67; G16H 50/30

(57) ABSTRACT

An example system includes communication circuitry configured to communicate with a medical device system, memory communicatively coupled to the communication circuitry and being configured to store an identifier associated with the medical device system and a subscription service level of a patient, and processing circuitry communicatively coupled to the communication circuitry and the memory. The processing circuitry is configured to control the communication circuitry to receive a communication associated with the medical device system, the communication comprising an identifier. The processing circuitry is configured to determine, based on the identifier, the subscription service level of the patient. The processing circuitry is configured to generate, based on the subscription service level of the patient, a configuration message and control the communication circuitry to transmit the configuration message to the medical device system.

This application claims the benefit of U.S. Provisional Application Ser. No. 63/267,825, filed Feb. 10, 2022, which is entitled “FEATURE SUBSCRIPTIONS FOR MEDICAL DEVICE SYSTEM FEATURE SETS” and is hereby incorporated by reference in its entirety.

### FIELD

This disclosure generally relates to systems including medical devices and, more particularly, to configuring features of such systems.

### BACKGROUND

A variety of devices may be configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect health events, including acute health events, based on the physiological signals, such as episodes of cardiac arrhythmia, myocardial infarction, stroke, falls, or seizure. Such physiological signals may include cardiac related signals, as well as signals related to comorbidities, like glucose levels. Example arrhythmia types include cardiac arrest (e.g., asystole), ventricular tachycardia (VT), and ventricular fibrillation (VF). The devices may store ECG and other physiological signal data collected during a time period including an episode as episode data. Such acute health events may be associated with significant rates of death, particularly if not treated quickly.

For example, VF and other malignant tachyarrhythmias are the most commonly identified arrhythmia in sudden cardiac arrest (SCA) patients. If this arrhythmia continues for more than a few seconds, it may result in cardiogenic shock and cessation of effective blood circulation. The survival rate from SCA decreases between 7 and 10 percent for every minute that the patient waits for defibrillation. Consequently, sudden cardiac death (SCD) may result in a matter of minutes.

### SUMMARY

In general, the disclosure describes techniques for affecting configuration of one or more devices of a medical device system to alter the functionality of the one or more devices of the medical device system. For example, a patient having a medical device, such as an implantable medical device, a wearable medical device, or the like, may subscribe to a health monitoring service at a particular subscription service level. There may be a plurality of subscription service levels available, for example there may be two, three, four, or any number of subscription service levels available. The subscription service level may be indicative of a quality of service of monitoring. As used herein “quality of service” may include differentiated sets of functionality that may be tailored based on patient preferences, physician recommendations for appropriate level of care, cost of care, pre-clinical trial functionality, and/or other potential drivers of differentiated features that may be needed or desired, or are appropriate for one patient, but not another, or one type of patient, but not another type of patient. For example, a given subscription service level may enable more, less, or different features or functionality of a medical device system than a different subscription service level.

The medical device system may send a message to the health monitoring service which may include an identifier that is associated with the patient and/or medical device system. The health monitoring service may determine the subscription service level based on the identifier. The health monitoring service may generate and send a configuration message, based on the subscription service level of the patient, to the medical device system which may then use the configuration message to configure the functionality of one or more devices of the medical device system.

For example, the configuration message may be used by a particular device of to expand or limit the number or type of devices with which the particular device may communicate, or to provide other functionality, such as running a self-test, providing enhanced fidelity to measurements obtained by sensors, providing broader access to data sensed by sensors, providing enhanced reporting based on data sensed by sensors, providing enhanced alerts including more information, or the like. The configuration message may configure a device to act as a verification device to attempt to verify an acute health event, to provide medical assistance, to enlist a bystander to provide medical assistance, or the like. For example, the particular device may run a script or an application to configure the particular device based on configuration information in the configuration message.

By generating and sending configuration messages for configuring one or more devices of a medical device system, based on different subscription service levels, the techniques of this disclosure may facilitate the treatment of health episodes, the verification of suspected health episodes, or the like. As such, a patient may be more likely to receive appropriate treatment in a timely manner, which may lead to better patient outcomes. As the techniques of this disclosure include generating and sending a configuration message, which may be used to configure the functionality of one or more devices of a medical device system, the techniques of this disclosure may improve the functioning of medical systems including medical devices that may expand the number or type of devices with which the particular device may communicate, initiate a self-test, provide enhanced fidelity to measurements obtained by sensors, provide broader access to data sensed by sensors, provide enhanced reporting based on data sensed by sensors, provide enhanced alerts including more information, verify a medical condition, treat a medical condition, enlist a bystander to provide medical treatment, or the like. Such improvements to the functioning of medical systems may improve patient outcomes.

Unlike conventional systems, where a health monitoring service may not generate and send configuration messages and the functionality of devices of a medical device system may be relatively stagnant and uncoordinated, the techniques and systems of this disclosure may provide for expanded functionality options and coordinated responses by, for example, a plurality of devices of the medical device system, to an acute health event. For example, the techniques of this disclosure may be utilized to configure a plurality of devices of the medical device system to attempt to validate an acute health event. Such devices may therefore work in a more coordinated manner than individually programmed devices. Additionally, by generating and sending a configuration message, based on a subscription service level, the techniques of this disclosure provide a user with the ability to select a level of configurability of the medical device system.

In one example, a computing system includes communication circuitry configured to communicate with one or more devices of a medical device system, the medical device system comprising at least an implantable medical device (IMD); memory communicatively coupled to the communication circuitry and being configured to store an identifier associated with the medical device system and a subscription service level of a patient; and processing circuitry communicatively coupled to the communication circuitry and the memory, the processing circuitry being configured to: control the communication circuitry to receive a communication associated with the medical device system, the communication comprising an identifier associated with the medical device system; determine, based on the identifier, the subscription service level of the patient, the subscription service level of the patient being indicative of a quality of service for the patient; generate, based on the subscription service level of the patient, a configuration message, the configuration message comprising information for configuring at least one device of the medical device system to affect the functionality of the at least one device; and control the communication circuitry to transmit the configuration message to the medical device system.

In another example, a method includes receiving, by communication circuitry, a communication associated with a medical device system, the communication comprising an identifier associated with the medical device system; determining, by processing circuitry and based on the identifier, a subscription service level of a patient associated with the medical device system, the subscription service level being indicative of a quality of service for the patient; generating, by the processing circuitry and based on the subscription service level of the patient, a configuration message, the configuration message comprising information for configuring at least one device of the medical device system to affect the functionality of the at least one device; and transmitting, by the communication circuitry, the configuration message to the medical device system.

In another example, a non-transitory computer-readable storage medium stores instructions that, when executed, cause processing circuitry to control communication circuitry to receive a communication associated with a medical device system, the communication comprising an identifier associated with the medical device system; based on the identifier, determine a subscription service level of a patient associated with the medical device system, the subscription service level being indicative of a quality of service for the patient; based on the subscription service level of the patient, generate a configuration message, the configuration message comprising information for configuring at least one device of the medical device system to affect the functionality of the at least one device; and control the communication circuitry to transmit the configuration message to the medical device system.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured detect health events of a patient, and to respond to such detections, in accordance with one or more techniques of this disclosure.

FIG. 2 is a block diagram illustrating an example configuration of a patient sensing device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flow diagram illustrating subscription service level techniques according to the present disclosure.

FIG. 6A is a perspective drawing illustrating an insertable cardiac monitor.

FIG. 6B is a perspective drawing illustrating another insertable cardiac monitor.

Like reference characters refer to like elements throughout the figures and description.

### DETAILED DESCRIPTION

A variety of types of implantable and computing devices are configured detect arrhythmia episodes and other health events, including acute health events, based on sensed ECGs and, in some cases, other physiological signals. Computing devices that may be used to non-invasively sense and monitor ECGs and other physiological signals include wearable devices with electrodes configured to contact the skin of the patient, such as patches, watches, or necklaces, and other non-contact monitoring devices, such as devices configured to monitor sound, radar, light, images, etc. Such computing devices may facilitate relatively longer-term monitoring of patient health during normal daily activities.

Implantable medical devices (IMDs), external medical devices (e.g., insulin pumps, left ventricle assist devices, etc.), and wearable devices, may also sense and monitor ECGs and other physiological signals (which may include cardiac-related signals, and/or blood pressure, glucose levels or other physiological signals), and detect health events, including acute health events, such as episodes of arrhythmia, cardiac arrest, myocardial infarction, falls, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ Insertable Cardiac Monitor (ICM) or the LINQ II™ ICM, available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network. While the techniques of this disclosure are discussed primarily with respect to cardiac IMDs and cardiac health, these techniques may be applied to other devices, including other IMDs and/or wearable devices which may be used to monitor other physiological parameters (e.g., non-cardiac) indicative of the health of the patient.

An IMD may interact with other devices. The IMD may communicate with an external device, such as a smart phone, and the communication may include an identifier associated with the IMD or patient to the external device. The external device may communicate with a remote health monitoring system, including the identifier. The health monitoring system may determine a subscription service level based on the identifier and provide a configuration message back to the external device or IMD to affect a configuration of the external device or IMD to alter the functionality of the external device or IMD. By providing for the configuration of a medical device system based on a subscription service level, a health monitoring system may provide different levels of functionality thereby facilitating a quality of service that may improve patient outcomes. In some examples, the subscription service level may be associated with a recurring payment. In some examples, the subscription service level may be a prepaid subscription service level, for example added on to or included with the cost of a device. In some examples, the subscription service level may be a charge per instance subscription service level. In some examples, the subscription service level may be a charge per continuous use subscription service level.

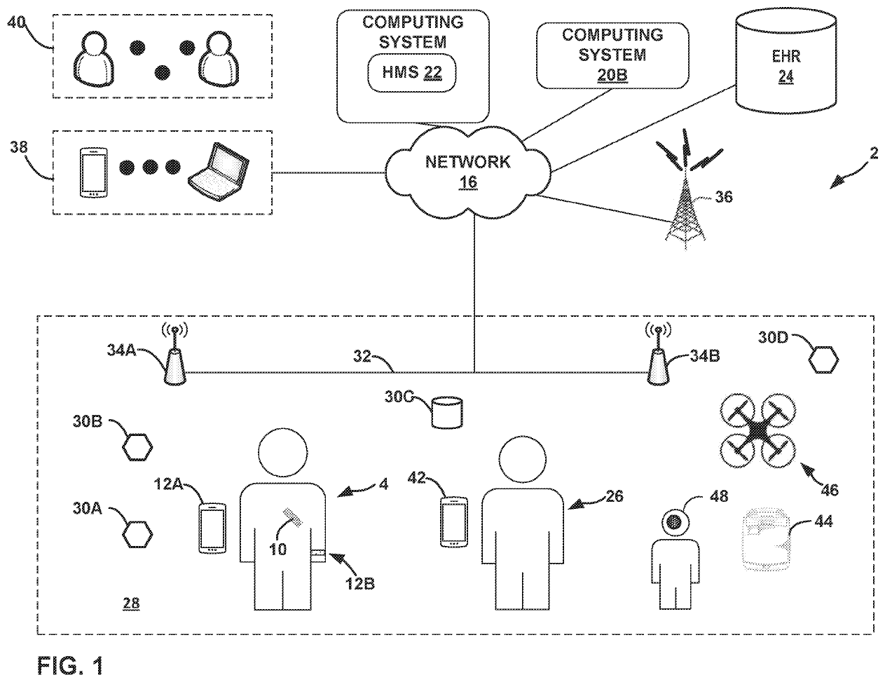

FIG. 1 is a block diagram illustrating an example system 2 configured detect health events, e.g., acute health events, of a patient 4, and to respond to such detection, in accordance with one or more techniques of this disclosure. While system 2 is depicted as including certain devices, a system, such as system 2, may include fewer, more, or different devices and still fall within the scope of this disclosure. As used herein, the terms “detect,” “detection,” and the like may refer to detection of an acute health event presently (at the time the data is collected) being experienced by patient 4, as well as detection based on the data that the condition of patient 4 is such that they have a suprathreshold likelihood of experiencing the event within a particular timeframe, e.g., prediction of the acute health event. Although described primarily in the context of detection of acute health events, the techniques implemented by system 2 may more generally relate to collection and communication of health data of patient 4, in some examples. The example techniques may be used with one or more patient sensing devices, e.g., IMD 10, which may be in wireless communication with one or more patient computing devices, e.g., patient computing devices 12A and 12B (collectively, “computing devices 12”). Although not illustrated in FIG. 1, IMD 10 include electrodes and other sensors to sense physiological signals of patient 4, and may collect and store sensed physiological data based on the signals and detect events based on the data. While this disclosure primarily discusses a sensing device as being an implantable medical device, IMD 10, in some cases the sensing device may be implemented as an external medical device or a wearable device rather than an IMD. As such, the techniques discussed herein may be practiced by an external medical device or a wearable device as well and the functionality attributed to IMD 10 may be practiced by an external medical device or a wearable device and still fall within the scope of this disclosure.

In some examples, although not depicted in FIG. 1, patient 4 may have a plurality of patient sensing devices, such as IMD 10. In some examples, the plurality of patient sensing devices may communicate with each other and/or computing device(s) 12. In some examples, the plurality of patient sensing devices may use time matching techniques, such determining a difference in a clock of each patient sensing device and applying the difference when saving a time of a sensed indication of an acute health event, or use a common clock. In this manner, sensed indications of acute health events of different patient sensing devices may be synchronized. In another example, one patient sensing device may be configured as a master and other patient sensing devices may be configured as slaves.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a medical device system includes one or more patient sensing devices, which may be implanted within patient 4 or external to (e.g., worn by) patient 4.

The example of FIG. 1 includes environment 28. Environment 28 may be a home, office, or place of business, or public venue, as examples. Computing devices 12 may be configured for wireless communication with IMD 10. Computing devices 12 may retrieve or receive event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may include any computing device configured for wireless communication with IMD 10, such as a desktop, laptop, or tablet computer. Computing devices 12 may communicate with IMD 10 and each other according to the Bluetooth®, Bluetooth® Low Energy (BLE), or other wireless communication protocols, as examples. In some examples, only one of computing devices 12, e.g., computing device 12A, is configured for communication with IMD 10, e.g., due to execution of software (e.g., part of a health monitoring application as described herein) enabling communication and interaction with IMD 10 or due to permissions configured on IMD 10, for example, which may be based on a subscription service level of patient 4 of a health monitoring system (HMS 22). In some examples, computing device(s) 12 may be configured to receive an indication of an acute health event of patient 4 from IMD 10.

In some examples, computing device(s) 12, e.g., wearable computing device 12B in the example illustrated by FIG. 1A, may include electrodes and other sensors to sense physiological signals of patient 4, and may collect and store physiological data and detect episodes based on such signals. Computing device 12B may be incorporated into the apparel of patient 4, such as within clothing, shoes, eyeglasses, a watch, ring, necklace, wristband, a hat, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 facilitates detection of acute health events of patient 4 by system 2, and the responses of system 2 to such acute health events.

Computing device(s) 12 may transmit data, including data retrieved or received from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

In some examples, HMS 22 (or another device, such as any of computing devices 12, or IoT devices 30) may implement a subscription service model. For example, HMS 22 may offer a plurality of subscription service levels to patients subscribing to a monitoring service of HMS 22. There may be a plurality of subscription service levels available, for example there may be two, three, four, or any number of subscription service levels available. For example, a given subscription service level may enable more, less, or different features or functionality of a medical device system than a different subscription service level.

A subscription service level may be indicative of a quality of service offered by HMS 22. For example, one subscription service level may include a higher quality of service than another. For example, a higher subscription service level may enable IMD 10 to communicate with a larger number of computing devices 12 than a lower subscription service level, which may limit the number of computing devices with which IMD 10 may communicate. For example, the lower subscription service level may limit IMD 10 to only communicate with computing device 12A, while the higher subscription service level may permit IMD 10 to communicate with computing device 12A, computing device 12B, Internet of Things (IoT) devices, such as IoT devices 30A-30D (collectively “IoT devices 30”), drone 46 and/or robot 48. For example, IoT devices 30 may include as examples smartphones, wearable devices, smart speakers, video cameras, infrared cameras, thermal cameras, radar systems, sonar systems, lidar systems, or bed sensors. In some examples, different ones or groups of IoT devices 30, drone 46, and/or robot 48 may be associated with different subscription service levels.

In some examples, a higher subscription service level may permit IMD 10 to communicate with additional devices while patient 4 is traveling. For example, if patient 4 is staying at a hotel having a smart television (which may be an example of an IoT device, such as IoT devices 30), the higher subscription service level may enable communications between IMD 10 and the smart television, in addition to computing devices 12, while the lower subscription service level may not enable such communications. In some examples, a higher subscription service level may facilitate more services while patient 4 is traveling. For example, a higher subscription service level may link up an emergency contact to an insurance provider, add international calling capabilities, or other services that may not be available at a lower subscription service level.

In some examples, when patient 4 begins subscribing to a monitoring service of HMS 22, IMD 10 may send a communication to computing device 12A. Such a communication may include an identifier associated with IMD 10 or patient 4. As IMD 10 is implanted in patient 4, an identifier associated with IMD 10 or with patient 4, is therefore associated with both IMD 10 and patient 4. Computing device 12A may in turn send a communication to HMS 22 via network 16 which may include the identifier. HMS 22 may determine, based on the identifier, a subscription service level of patient 4. For example, HMS 22 may look up the identifier in a lookup table in a database (not shown in FIG. 1) to determine the subscription service level for patient 4. The subscription service level may be indicative of a quality of service for the patient. HMS 22 may then transmit a configuration message based on the subscription service level of patient 4 to any of computing devices 12, IoT devices 30, drone 46, and/or robot 48, which may in turn transmit a configuration message to IMD 10. This configuration message may include information for configuring at least one device of system 2, such as IMD 10, computing devices 12, IoT devices 30, drone 46, robot 48, or the like, to affect the functionality of the at least one device. For example, the configuration message may be used by a particular device of IMD 10, computing devices 12, IoT devices 30, drone 26, or robot 48 to expand or limit the number or type of devices with which the particular device may communicate, or to provide other functionality, such as running a self-test, providing enhanced fidelity to measurements obtained by sensors of system 2, providing broader access to data sensed by sensors of system 2, providing enhanced reporting based on data sensed by sensors of system 2, providing enhanced alerts including more information, or the like. For example, the particular device may run a script or an application to configure the particular device based on configuration information in the configuration message.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to detect acute health events of patient 4 based on data sensed by IMD 10 and, in some cases, other data, such as data sensed by computing devices 12A and/or 12B, data from EHR 24, data from IoT devices 30, data from drone 46, or data from robot 48. In response to detection of an acute health event, IMD 10 may wirelessly transmit an indication of the acute health event, such as a message, to one or both of computing devices 12A and 12B, or other devices, which may be based on the subscription service level of patient 4. The message may indicate that IMD 10 detected an acute health event of patient 4. The message may indicate a time that IMD 10 detected the acute health event. The message may include physiological data collected by IMD 10, e.g., data which lead to detection of the acute health event, data prior to detection of the acute health event, and/or real-time or more recent data collected after detection of the acute health event. The physiological data may include values of one or more physiological parameters and/or digitized physiological signals. Some examples of acute health events are cardiac arrest, ventricular fibrillation, ventricular tachycardia, myocardial infarction, pause in heat rhythm (asystole), pulseless electrical activity (PEA), acute respiratory distress syndrome (ARDS), stroke, seizure, fall, anaphylactic shock, or respiratory failure.

In some examples, IoT devices, such as IoT devices 30, drone 46, or robot 48 may be additional devices with which IMD 10 may communicate, either directly, or through at least on of computing devices 12 based on a subscription service level of patient 4. For example, if patient 4 has a higher subscription service level, IMD 10 and/or IoT devices 30, drone 46, and/or robot 48 may be configured to communicate with each other, whereas if patient 4 has a lower subscription service level, IMD 10 may communicate with fewer of or none of IoT devices 30, drone 46, and/or robot 48.

In some examples, IoT devices 30, drone 46, or robot 48 may be configured to act as verification devices or part of a verification system. The verification device or system may attempt to verify the acute health event. For example, the verification device or system may determine physiological parameters of patient 4 and may determine whether the physiological parameters of the patient meet predefined criteria. The predefined criteria may be indicative of the acute health event. In some examples, the use of the verification device or system may be based on a subscription service level of patient 4.

In some examples, a subscription service level may be a pay-per-use model in which patient 4 is billed for each usage event. For example, it patient 4 desires to have IMD 10 communicate with IoT devices 30 for a particular time or under a particular set of circumstances (e.g., physiological parameters of patient 4 indicate that a health event is occurring), HMS 22 may bill patient 4 for such usage. In some examples, patient 4 may be billed for IMD 10 communicating with an emergency medical service, for example, when IMD 10 senses a health event of patient 4 occurring, whether directly, or via any of computing devices 12, IoT devices 30, HMS 22, or the like.

In some examples, based on the subscription service level, IMD 10 may perform a self-test to verify that IMD 10 is performing as expected. For example, if patient 4 has a pay-per-use subscription service level, IMD 10 may perform a self-test when patient 4 initiates a self-test via one of computing devices 12 or IoT devices 30, and HMS 22 may bill patient for configuring IMD 10 to perform the self-test. In some examples, under a higher subscription service level, patient 4 may be enabled to control IMD 10 to perform a self-test whenever patient 4 desires.

In some examples, a subscription service level may be tailored to an individual patient, such as patient 4. For example, patient 4 may have a relatively unique health profile, with their own disease states and comorbidities, which may indicate a risk of a health event of patient 4. In some examples, HMS 22 may retrieve information from EHR 24 regarding the health profile of patient 4 and, based on the health profile of patient 4, suggest to patient 4 a subscription service level based on the health profile of patient 4. For example, HMS may communicate with computing device 12A or 12B or another device to suggest to patient 4 the subscription service level. In some examples, HMS 22 may be configured, based on a subscription service level of patient 4, to send an individualized patient risk report, which may include sensed physiological parameters of patient 4, a physician's analysis of such parameters, or other information based on physiological parameters of patient 4 sensed by IMD 10 or other devices of system 2.

In some examples, based on the subscription service level, IMD 10 may send notifications to one or more computing devices 12 or IoT devices 30, including those of patient associated responders, when IMD 10 provides treatment or therapy, such as delivering a shock to a heart of patient 4. In some examples, IMD 10 may, based on the subscription service level, send notifications to one or more computing devices 12 or IoT devices 30 (including those of patient associated responders), drone 46, robot 48, or an emergency medical service, when IMD 10 or another device of system 2 senses a health event, such as tachycardia, elevated blood glucose levels, a seizure, a stroke, or the like. In some examples, based on the subscription service level of patient 4, IMD 10 may send data to devices of patient associated responders (also referred to herein as “patient associated responder devices”) such as friends, family members, caregivers, or the like, verifying the functionality of IMD 10 and/or other devices of system 2. For example, IMD 10 may perform a self-test and send a message indicative of whether the self-test was passed or failed. In some examples, based on the subscription service level of patient 4, the message may include further information, such as which portion of the self-test failed, how well IMD 10 performed during the self-test or the like. In some examples, based on the subscription service level of patient 4, IMD 10 may send a message to patient associated responder devices indicative of an amount of time or a percentage of time during which IMD 10 is in communication with computing device 12A or another device of system 2. In some examples, according to the subscription service level of patient 4, system 2 may enable IMD 10 to send data to a monitoring service, such as HMS 22, to monitor the working condition of IMD 10 and/or the connectivity status of IMD 10 with other devices of system 2.

In some examples, based on a subscription service level, HMS 22 may send a configuration message to computing devices 12, IoT devices 30, drone 46, and/or robot 48 to configure a mesh network of computing devices 12, IoT devices 30, drone 46, and/or robot 48 permitting IMD 10 to quickly communicate through the mesh network to an emergency medical service, a clinician, a caregiver, a bystander, or the like. As such, patient 4 may receive medical treatment in an urgent manner.

In some examples, under a higher subscription service level, IMD 10 may be configured to communicate with patient associated responder devices, such as computing devices 12 or IoT devices 30 of patient associated responders (such as caregivers, friends, family members, or other people who may be nearby) in the event that IMD 10 senses a health event of patient 4. In some examples, under a higher subscription service level, IMD 10 may be configured to communicate with devices, such as computing devices 12 or IoT devices 30 of patient associated responders in a test mode to verify that IMD 10 can communicate with computing devices 12 or IoT devices 30 of patient associated responders during a health event of patient 4.

For example, environment 28 may include a plurality of cameras (e.g., any of IoT devices 30). The plurality of cameras may be located throughout a house, for example, and may be used to determine a location of patient 4 and/or to verify the acute health event. In some examples, one or more cameras are only activated by the verification device or system for locating patient 4 or verifying the acute health event in certain instances. For example, a camera may only be activated if the sensed indication of the acute health event is sufficiently strong. For example, strength of the indication may be scored by IMD 10 or computing device(s) 12, and the camera may only be activated if the score is equal to or higher than a predetermined score. In some examples, such features may be based on a subscription service level of patient 4.

In some examples, environment 28 includes a house. IoT devices 30 may determine whether patient 4 is inside the house or outside the house. For example, IoT devices 30 may include a smart speaker, smart lock(s), and/or cameras that may monitor a location of patient 4. Processing circuitry of IoT devices 30, computing devices 12, and/or IMD 10 may determine the location of patient 4 based on the monitoring. The processing circuitry may use the determined location to dispatch drone 46 and/or robot 48 to the location of patient 4. In some examples, such features may be based on a subscription service level of patient 4.

In some examples, drone 46 or robot 48 may navigate to the proximity of patient 4 to determine physiological parameters of patient 4 or to treat patient 4. In some examples, cameras may capture video, infrared, thermal, or other images of patient 4. The images may be processed to identify physiological parameters of patient 4. For example, system 2 may determine posture and facial features of patient 4 through the use of captured images. In some examples, the system 2 may use facial recognition techniques on one or more images to detect changes in a face of patient 4, such as a droop, that may be a side effect of a stroke.

Infrared cameras, radar systems, sonar systems, lidar systems or other sensors may measure the presence or absence of a pulse, oxygen saturation levels, or breathing of patient 4 when attempting to verify the acute health event. The absence of a pulse, reduced oxygen saturation, the absence of breathing, or convulsions may each be used to verify the acute health event. For example, one of more of IoT devices 30 may be configured to sense heartbeat sounds and the verification device or system may use the heart beat sounds to determine a heartbeat or pulse of patient 4. In some examples, system 2 may use radar, lidar, sonar, bed sensors, or cameras to determine respiration rate of patient 4 or changes in surfaces of patient 4, for example, by monitoring changes in the position of chest of patient 4 over time. In some examples, system 2 may use a camera to sense blood flow in patient 4 (e.g., using a red-sensitive image of a face of patient 4, or other exposed skin of patient 4). In other examples, system 2 may use radar, sonar, or lidar to identify area of interest on patient 4 and analyze a sequence of images from a camera over time to sense blood flow in patient 4. In some examples, system 2 may include an oxygen sensor, which may be integrated into computing device 12B, for example, which may be configured to monitor an oxygen saturation level of blood of patient 4. In some examples, system 2 may use traditional signal processing or machine learning techniques to combine measurements from multiple sensors to verify the acute health event. In some examples, such features may be based on a subscription service level of patient 4.

In some examples, in response to the message indicative of the sensing of the acute health event from IMD 10, computing device(s) 12 may prompt patient 4 to provide a response. For example, computing device(s) 12 may audibly ask the patient if they are okay or may ask the patient to provide tactile input indicating that they are okay in an attempt to elicit a response. Computing device(s) 12 may wait for a response from patient 4. After a predetermined period of time, which may be on the order of up to 60 seconds, if the patient had not responded, computing device(s) 12 may attempt to verify the acute health event or contact an emergency medical service. For example, computing device 12B may attempt to take a pulse of patient 4. In some examples, such features may be based on a subscription service level of patient 4.

Other devices in the environment 28 of patient 4 may also be configured to verify the acute health event. For example, environment 28 may include one or more IoT devices 30 illustrated in the example of FIG. 1. IoT devices 30 may include, as examples, so called “smart” speakers, smart televisions, cameras, lights, locks, thermostats, appliances, actuators, controllers, or any other smart home (or building) devices. For example, IoT devices 30 may include video cameras, infrared cameras, thermal cameras, radar systems, sonar system, lidar systems, bed sensors, smart speaker, smart television, or other IoT devices. These IoT devices 30 may be configured to determine physiological parameters of patient 4. For example, IoT devices 30 may be configured to determine at least one of pulse rate, blood perfusion, breathing rate, breathing intensity, posture, facial features, color of a face, or an electrocardiogram. In some examples, such features may be based on a subscription service level of patient 4.

In some examples, IoT devices 30 or computing device(s) 12 may be configured to determine a location of patient 4. For example, a camera may capture an image and image processing techniques may be used to determine that patient 4 is in a captured image. A radar, sonar, or lidar system may transmit radio, sound, or light waves, respectively, and measure reflections of those waves to determine a location of patient 4. Bed sensors may determine that patient 4 is in bed based on pressure placed on the bed sensors. In some examples, computing device(s) 12 may receive, from IoT devices 30, information indicative of the location of patient 4 and may process such information to determine the location of patient 4. In some examples, such features may be based on a subscription service level of patient 4.

As mentioned above, in some examples, IoT devices 30 may include bed sensors. Bed sensors may be useful in verifying the acute health event as lethal acute health events frequently occur during sleep. In some examples, IoT devices 30 may include a device, such as a camera, that is configured to monitor the behavior of patient 4. For example, the device may capture images of patient 4 and processing circuitry may perform image analysis to determine a location of patient 4 or physiological parameters of patient 4. In some examples, IoT devices 30 may include a device, such as a radar system, that is configured to monitor sleep apnea of patient 4. For example, a radar system, may transmit radio waves and measure reflections of the radio waves to monitor sleep apnea. Reflections of the radio waves may show relatively consistent movement of the chest of patient 4 when breathing normally. Reflections of the radio waves may show more erratic movement of the chest of patient 4 during a sleep apnea episode, where patient 4 may pause breathing and then gasp for air.

In the example of FIG. 1, IoT device 30C is a smart speaker and/or controller, which may include a display. In some examples, rather than computing device(s) 12 attempting to solicit the response from patient 4, IoT device 30C may attempt to solicit the response. IoT devices 30 may provide audible and/or visual alarms when configured with output devices to do so. As other examples, IoT devices 30 (or computing device(s) 12) may cause smart lights throughout environment 28 to flash or blink and unlock or open smart locks on doors of the house. By opening smart locks, IoT devices 30 or computing device(s) 12 may facilitate quick entry into the home by EMS personnel or bystander 26. In some examples, IoT devices 30 that include cameras or other sensors may activate those sensors to collect data regarding patient 4, e.g., for evaluation or verification of the condition of patient 4 and, in some examples, a location of patient 4.

In some examples, drone 46 may be an unmanned aerial vehicle (UAV). Drone 46 may be equipped with a number of sensors and/or actuators to perform a number of operations, such as determine physiological parameters of patient 4. For example, drone 46 may include a camera or other sensors to navigate to its intended location, identify patient 4 and, in some cases, bystander 26, and to evaluate or verify a condition of patient. In some examples, drone 46 may be configured to determine the location of patient 4, such as through camera images captured by drone 46 or other techniques. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., automated external defibrillator (AED) 44, and/or medication to the location of patient 4. In some examples, deployment of drone 46 is based on a subscription service level of patient 4.

In some examples, robot 48 may be equipped with a number of sensors and/or actuators to perform a number of operations, such as determine physiological parameters of patient 4. For example, robot 48 may include a camera or other sensors to navigate to its intended location, identify patient 4 and, in some cases, bystander 26, and to evaluate a condition of patient. In some examples, robot 48 may be configured to determine the location of patient 4, such as through camera images captured by robot 48, audio captured by robot 48, or other techniques. In some examples, robot 48 may include user interface devices to communicate with patient 4 and/or bystander 26. In some examples, robot 48 may provide directions to bystander 26, to the location of patient 4 and regarding how to provide first responder care, such as CPR, to patient 4. In some examples, robot 48 may carry or include medical equipment, e.g., AED 44, and/or medication to the location of patient 4. In some examples, robot 48 may perform a medical intervention on patient 4, such as perform CPR or defibrillation on patient 4, or perform some other medical treatment. In some examples, deployment of robot 48 is based on a subscription service level of patient 4.

In some examples, in response to the message from IMD 10, and based on a subscription service level of patient 4, computing device(s) 12 may also output an alarm that may be visual and/or audible, and configured to immediately attract the attention of patient 4 or any person in environment 28 with patient 4, e.g., a bystander 26. Computing device(s) 12 may also transmit a message to HMS 22 via network 16. The message may include the data received from IMD 10 and, in some cases, additional data collected by computing device(s) 12 or other devices in response to the detection of the acute health event by IMD 10. For example, the message may include a location of patient 4 determined by computing device(s) 12, IoT devices 30A-30D, drone 46, or robot 48.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device(s) 12 as described above. In some examples, IMD 10 is configured to communicate wirelessly with one or more of IoT devices 30, e.g., in response to detection of an acute health event when communication with computing devices 12 is unavailable or not preferred or based on a subscription service level of patient 4. In some examples, IoT device(s) 30 may be configured to provide some or all of the functionality ascribed to computing devices 12 herein.

Environment 28 includes computing facilities, e.g., a local network 32, by which IMD 10, computing devices 12, IoT devices 30, and other devices within environment 28 may communicate via network 16, e.g., with HMS 22. For example, environment 28 may be configured with wireless technology, such as 802.11 wireless networks, 802.15 ZigBee networks, an ultrawideband protocol, near-filed communication, or the like. Environment 28 may include one or more wireless access points, e.g., wireless access points 34A and 34B (collectively, “wireless access points 34”) that provide support for wireless communications throughout environment 28. Additionally, or alternatively, e.g., when local network is unavailable, IMD 10, computing devices 12, IoT devices 30, and other devices within environment 28 may be configured to communicate with network 16, e.g., with HMS 22, via a cellular base station 36 and a cellular network.

Computing device(s) 12, and in some examples IoT devices 30, may include input devices and interfaces to allow a user to override the alarm in the event the detection of the acute health event by IMD 10 was false. In some examples, such functionality may be based on a subscription service level of patient 4. In some examples, one or more of computing device(s) 12 and IoT device(s) 30 may implement an event assistant. The event assistant may provide a conversational interface or a tactile interface for patient 4 and/or bystander 26 to exchange information with a computing device or IoT device. The event assistant may query the user regarding the condition of patient 4 in response to receiving the alert message from IMD 10. Responses from the user may be used to confirm or override detection of the acute health event by IMD 10, or to provide additional information about the acute health event or the condition of patient 4 more generally that may improve the efficacy of the treatment of patient 4. For example, information received by the event assistant may be used to provide an indication of severity or type (differential diagnosis) for the acute health event. The event assistant may use natural language processing and context data to interpret utterances by the user. In some examples, in addition to receiving responses to queries posed by the assistant, the event assistant may be configured to respond to queries posed by the user. For example, patient 4 may indicate that they feel dizzy and ask the event assistant, “how am I doing?”.

In some examples, computing device(s) 12 and/or HMS 22 may implement one or more algorithms to evaluate the sensed physiological data received from IMD 10, and in some cases additional physiological or other data sensed or otherwise collected by the computing device(s) or IoT devices 30, to confirm or override the detection of the acute health event by IMD 10. In some examples, computing device(s) 12 and/or computing system(s) 20 may have greater processing capacity than IMD 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the acute health event. In some examples, such functionality may be based on a subscription service level of patient 4.

In examples in which computing device(s) 12 are configured to perform an acute health event confirmation analysis or verification, computing device(s) 12 may transmit alert messages to HMS 22 and/or IoT devices 30 in response to confirming or verifying the acute health event. In some examples, computing device(s) 12 may be configured to transmit the alert messages prior to completing the confirmation or verification analysis, and transmit cancellation messages in response to the analysis overriding the detection of the acute health event by IMD 10. In some examples, the ability to override the detection of the acute health event may be based on the subscription service level of patient 4. HMS 22 may be configured to perform a number of operations in response to receiving an alert message from computing device(s) 12 and/or IoT device(s) 30. HMS 22 may be configured to cancel such operations in response to receiving a cancellation message from computing device(s) 12 and/or IoT device(s) 30.

In some examples, computing device(s) 12 may transmit the alert to a care provider, an emergency medical technician, or other designated persons in environment 28 or near environment 28. For example, the alert may be a communication to the emergency medical technician, or local neighborhood alert system with an automated emergency defibrillator service, to a care provider, etc. In some examples, the alert includes collected data from IMD 10 and the verification device or system, such that medical personnel may be prepared to take quick action on arrival in environment 28. In some examples, the alert includes at least one of a telephone call, a short message service message, an email, a web alert, a security system alert, a social media alert, an audible alert, or a visual alert. In some examples, the timing, the content, and/or the recipient of the alert may be based on a subscription service level of patient 4.

In some examples, the verification device or system may send an alert through a security system (which may be one of IoT devices 30) in environment 28 to flash a “save our souls” (SOS) message, sound an audible alarm, or the like. In some examples, the verification device or system may notify neighbors of patient 4 of a medical emergency. In some examples, the verification system may send an alarm or warning to everyone and every device around the environment 28. For example, the verification device or system may send an audible warning to IoT device 30C (the smart speaker). In some examples, the verification device or system may send an alarm to a social media group or group email, for example, where there is a geographic or therapy relevance to patient 4.

For example, HMS 22 may be configured to transmit alert messages to one or more computing devices 38 associated with one or more care providers 40 via network 16, based on a subscription service level of patient 4. Care providers may include emergency medical systems (EMS) and hospitals, and may include particular departments within a hospital, such as an emergency department, catheterization lab, or a stroke response department. Computing devices 38 may include smartphones, desktop, laptop, or tablet computers, or workstations associated with such systems or entities, or employees of such systems or entities. The alert messages may include any of the data collected by IMD 10, computing device(s) 12, and IoT device(s) 30, including sensed physiological parameters, time of the acute health event, location of patient 4, and results of the analysis by IMD 10, computing device(s) 12, IoT device(s) 30, and/or HMS 22. The information transmitted from HMS 22 to care providers 40 may improve the timeliness and effectiveness of treatment of the acute health event of patient 4 by care providers 40. In some examples, instead of or in addition to HMS 22 providing an alert message to one or more computing devices 38 associated with an EMS care provider 40, computing device(s) 12 and/or IoT devices 30 may be configured to automatically contact EMS, e.g., autodial 911 (e.g., in the United States or North America to use the telephone system to contact a 911 call center), in response to receiving an alert message from IMD 10. Again, such operations may be canceled by patient 4, bystander 26, or another user via a user interface of computing device(s) 12 or IoT device(s) 30, or automatically cancelled by computing device(s) 12 based on a confirmatory analysis or verification performed by the computing device(s) overriding the detection of the acute health event by IMD 10. In some examples, the ability to cancel may be based on the subscription service level of patient 4. In some examples, there may be multiple tiers of the ability to cancel such operations based on the subscription service level of patient 4. For example, with a first subscription service level, patient 4 may cancel the operations, while with a second subscription service level, patient 4 and/or bystander 26 and/or other early responder may cancel the operations.

Similarly, HMS 22 (or another device of system 2, such as any of computing devices 12 or IoT devices 30) may be configured to transmit an alert message to computing device 42 of bystander 26, which may improve the timeliness and effectiveness of treatment of the acute health event of patient 4 by bystander 26, based on a subscription service level of patient 4. Computing device 42 may be similar to computing devices 12 and computing devices 38, e.g., a smartphone. In some examples, HMS 22 may determine that bystander 26 is proximate to patient 4 based on a location of patient 4, e.g., received from computing device(s) 12, and a location of computing device 42, e.g., reported to HMS 22 by an application implemented on computing device 42. In some examples, HMS 22 may transmit the alert message to any computing devices 42 in an alert area determined based on the location of patient 4, e.g., by transmitting the alert message to all computing devices in communication with base station 36.

In some examples, the alert message to bystander 26 may be configured to assist a layperson in treating patient 4. For example, the alert message to bystander 26 may include a location (and in some cases a description) of patient 4, the general nature of the acute health event, directions for providing care to patient 4, such as directions for providing cardio-pulmonary resuscitation (CPR), a location of nearby medical equipment for treatment of patient 4, such as an AED 44 or life vest, and instructions for use of the equipment. In some examples, computing device(s) 12, IoT device(s) 30, and/or computing device 42 may implement an event assistant configured to use natural language processing and context data to provide a conversational interface for bystander 26. The assistant may provide bystander 26 with directions for providing care to patient 4, and respond to queries from bystander 26 about how to provide care to patient 4.

In some examples, HMS 22 may mediate bi-directional audio (and in some cases video) communication between care providers 40 and patient 4 or bystander 26. Such communication may allow care providers 40 to evaluate the condition of patient 4, e.g., through communication with patient 4 or bystander 26, or through use of a camera or other sensors of the computing device or IoT device, in advance of the time they will begin caring for the patient, which may improve the efficacy of care delivered to the patient. Such communication may also allow the care providers to instruct bystander 26 regarding first responder treatment of patient 4. In some examples, such features may be based on a subscription service level of patient 4.

In some examples, HMS 22 may control dispatch of a drone 46 to environment 28, or a location near environment 28 or patient 4. Drone 46 may be an unmanned aerial vehicle (UAV). Drone 46 may be equipped with a number of sensors and/or actuators to perform a number of operations. For example, drone 46 may include a camera or other sensors to navigate to its intended location, identify patient 4 and, in some cases, bystander 26, and to evaluate a condition of patient. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., AED 44, and/or medication to the location of patient 4. In some examples, such features may be based on a subscription service level of patient 4.

In some examples, HMS 22 may control dispatch of a robot 48 to environment 28, or a location near environment 28 or patient 4. Robot 48 may be equipped with a number of sensors and/or actuators to perform a number of operations. For example, robot 48 may include a camera or other sensors to navigate to its intended location, identify patient 4 and, in some cases, bystander 26, and to evaluate a condition of patient, such as taking an ECG or measuring a pulse. In some examples, robot 48 may act as an AED by touching two parts of the body of patient 4 with extendable arms having electrodes. In some examples, robot 48 may include user interface devices to communicate with patient 4 and/or bystander 26. In some examples, robot 48 may provide directions to bystander 26, to the location of patient 4 and regarding how to provide first responder care, such as CPR, to patient 4. In some examples, robot 48 may carry medical equipment, e.g., AED 44, and/or medication to the location of patient 4. In some examples, robot 48 may perform a medical intervention on patient 4, such as perform CPR or defibrillation on patient 4, or perform some other medical treatment. In some examples, such features may be based on a subscription service level of patient 4.

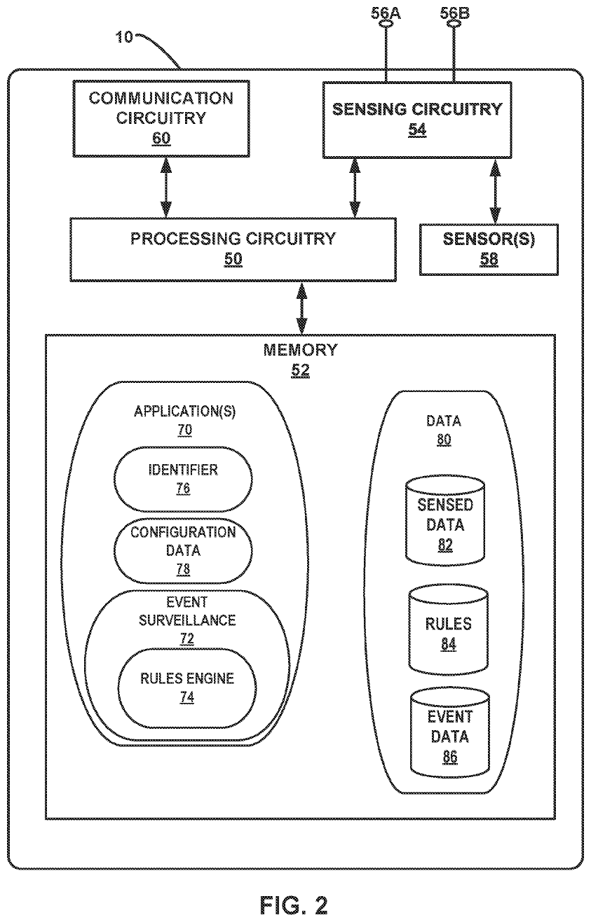

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), ferroelectric RAM (FRAM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode. In some examples, based on a subscription service level of patient 4, HMS 22 may provide to patient 4 via one of more of computing devices 12 or IoT devices 30, a report based on sensed physiological parameters of patient 4, such as the digitized ECG or other sensed physiological parameters.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes sensing circuitry 58, such as one or more accelerometers, microphones, optical sensors, temperature sensors, and/or pressure sensors. In some examples, sensing circuitry 54 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include an acute health event surveillance application 72. Processing circuitry 50 may execute event surveillance application 72 to detect an acute health event of patient 4 based on combination of one or more of the types of physiological data described herein, which may be stored as sensed data 82. In some examples, sensed data 82 may additionally include data sensed by other devices, e.g., computing device(s) 12, and received via communication circuitry 60. Event surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning.

In some examples, memory 52 may store an identifier 76 uniquely identifying either IMD 10 or patient 4. In such cases, identifier 76 may be said to be associated with IMD 10 and patient 4, as IMD 10 is implanted in patient 4. In some examples, memory 52 may store configuration data 78 associated with the subscription service level of patient 4. Processing circuitry 50 may use configuration data 78 to control functionality of IMD 10. For example, configuration data 78 may indicate which devices of computing devices 12 and IoT devices 30 with which IMD 10 may communicate, or provide other functionality, such as running a self-test, providing enhanced fidelity to measurements obtained by sensors of system 2, providing broader access to data sensed by sensors of system 2, providing enhanced reporting based on data sensed by sensors of system 2, providing enhanced alerts including more information, or the like. For example, IMD 10, any of computing devices 12, any of IoT devices 30, drone 46, and/or robot 48 may run a script or an application to configure itself based on configuration information in the configuration message.

As examples, event surveillance application 72 may detect a cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of a heart of patient 4 (FIG. 1) or other health event, such as high blood pressure or out-of-range glucose level. In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the patient has fallen based on data from an accelerometer alone, or in combination with other physiological data. When event surveillance application 72 detects a health event, including an acute health event, event surveillance application 72 may store the sensed data 82 that lead to the detection (and in some cases a window of data preceding and/or following the detection) as event data 86.

In some examples, in response to detection of an acute health event, processing circuitry 50 transmits, via communication circuitry 60, event data 86 for the event to computing device(s) 12 (FIG. 1). This transmission may be included in a message indicating the acute health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30. In response to receiving the message, computing device(s) 12 may attempt to elicit a response from patient 4 as discussed above with respect to FIG. 1. In some examples, such features may be based on a subscription service level of patient 4.

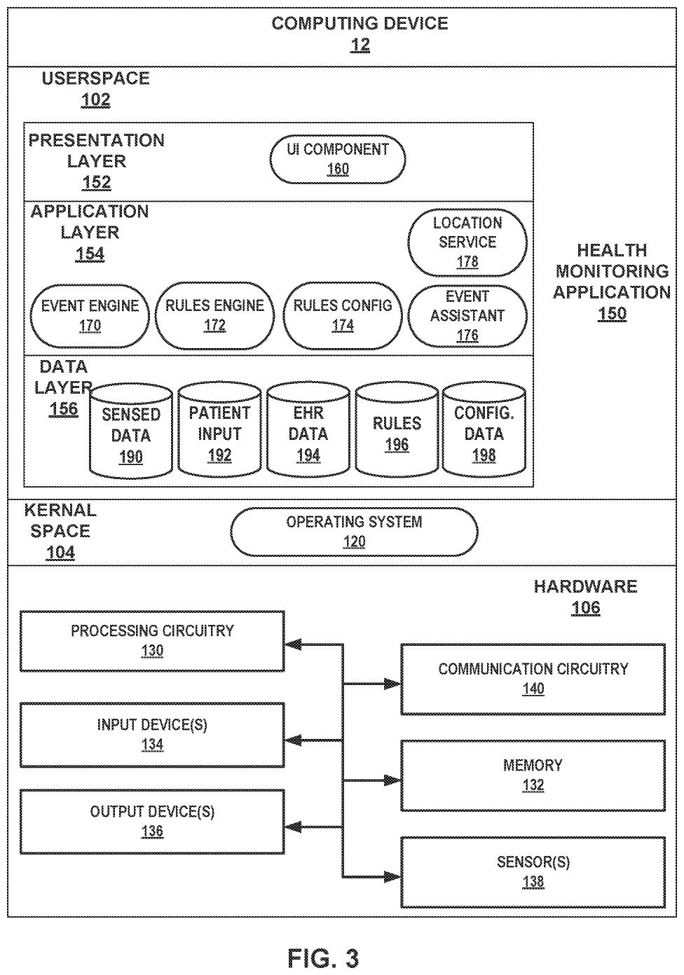

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30, drone 46, and robot 48 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, sensing circuitry 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random-access memories (RAM), dynamic random-access memories (DRAM), static random access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 136 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

Sensing circuitry 138 of computing device 12 may sense physiological parameters or signals of patient 4. Sensor(s) 138 may include electrodes, 3-axis accelerometers, an optical sensor, impedance sensors, temperature sensors, pressure sensors, heart sounds sensors, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE). In some examples, communications of communication circuitry 140 may be affected by configuration data 198. For example, if patient 4 has a higher subscription service level, communication circuitry 140 may be configured, through configuration data 198, to create a mesh network with other devices, and transmit a message indicative of a health event occurring in patient 4, for example, to an emergency medical service, via the mesh network.

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, rules engine 172, rules configuration component 174, event assistant 176, and location service 178. Event engine 170 may be responsive to receipt of an alert transmission from IMD 10 indicating that IMD 10 detected an acute health event. Event engine 170 may control performance of any of the operations in response to detection of an acute health event ascribed herein to computing device 12, such as activating an alarm, transmitting alert messages to HMS 22, controlling IoT devices 30, and analyzing data to confirm or override the detection of the acute health event by IMD 10. In some examples, such features may be configurable by configuration data 198 and may be based on a subscription service level of patient 4.

Rules engine 172 analyzes sensed data 190, and in some examples, patient input 192 and/or EHR data 194, to determine whether there is a sufficient likelihood that patient 4 is experiencing the acute health event detected by IMD 10 or to verify that patient 4 has experienced the acute health event detected by IMD 10. Sensed data 190 may include data received from IMD 10 as part of the alert transmission, additional data transmitted from IMD 10, e.g., in “real-time,” and physiological parameters and other data related to the condition of patient 4 collected by computing device(s) 12, IoT devices 30, drone 46, and/or robot 48. Rules engine 172 may determine whether the physiological parameters meet predefined criteria to verify the acute health event. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user, such as bystander 26. In some examples, queries and the ability to analyze patient input 192 in response to queries may be based on a subscription service level of patient 4. The queries and responses may occur responsive to the detection of the event by IMD 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

Rules engine 172 may apply rules 196 to the data. Rules 196 may include one or more models, algorithms, decision trees, and/or thresholds. In some cases, rules 196 may be developed based on machine learning. In some examples, rules 196 and the operation of rules engine 172 may provide a more complex analysis of the sensed data received from IMD 10, than is provided by rules engine 74 and rules 84. In some examples, rules 196 include one or more models developed by machine learning, and rules engine 172 applies feature vectors derived from the data to the model(s). For example, rules engine 172 may apply rules 196 to determine whether the physiological parameters captured by IMD 10 and the verification device or system meet predefined criteria to verify the acute health event.

Rules configuration component 174 may be configured to modify rules 196 (and in some examples rules 84) based on feedback indicating whether the detections and confirmations of acute health events by IMD 10 and computing device 12 were accurate. The feedback may be received from patient 4, or from care providers 40 and/or EHR 24 via HMS 22. In some examples, rules configuration component 174 may utilize the data sets from true and false detections and confirmations for supervised machine learning to further train models included as part of rules 196.

As discussed above, event assistant 176 may provide a conversational interface or tactile interface for patient 4 and/or bystander 26 to exchange information with computing device 12. Event assistant 176 may query the user regarding the condition of patient 4 in response to receiving the alert message from IMD 10. Responses from the user may be included as patient input 192. Event assistant 176 may use natural language processing and context data to interpret utterances by the user. In some examples, in addition to receiving responses to queries posed by the assistant, event assistant 176 may be configured to respond to queries posed by the user. In some examples, event assistant 176 may provide directions to and respond to queries regarding treatment of patient 4 from patient 4 or bystander 26.

Location service 178 may determine the location of computing device 12 and, thereby, the presumed location of patient 4. Location service 178 may use global position system (GPS) data, multilateration, and/or any other known techniques for locating computing devices. In some examples, location service 178 may utilize data from other devices to determine the location of patient 4, such as IoT devices 30, drone 46, or robot 48. For example, data from a camera, a radar system, a sonar system, or a lidar system may be used to determine where in environment 28 (FIG. 1) patient 4 is located. In some examples, location service 178 may track where patient 4 is during different times of the day and use the most frequent location at the time of the day that the indication of the acute medical event was sensed as a starting position to determine the location of patient 4. For example, location service 178 may employ one or more of the devices, such as IoT devices 30, drone 46, or robot 48, to check to see if patient 4 is located at the most frequent location for that time of day. In some examples, such features may be based on a subscription service level of patient 4.

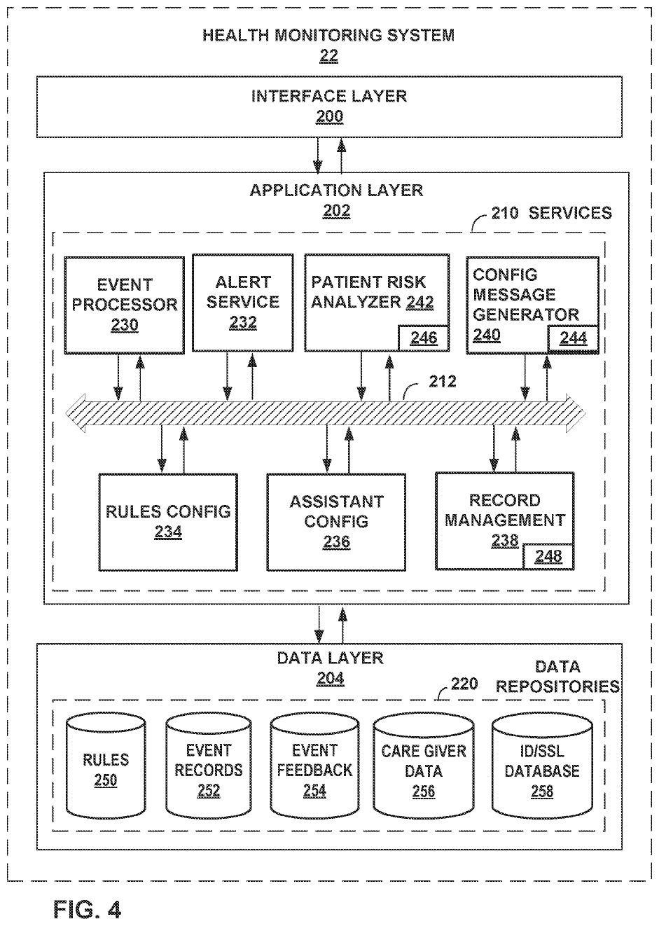

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software. While shown as a single unit, in some examples, HMS 22 may include one or more servers.

Computing devices, such as computing devices 12, IoT devices 30, computing devices 38, computing device 42, drone 46, and robot 48 may operate as clients that communicate with HMS 22 via interface layer 200. In some examples, which of such devices may communicate with HMS 22 may be based on a subscription service level of patient 4.

The computing devices typically execute client software applications, such as desktop application, mobile application, and web applications. Interface layer 200 represents a set of application programming interfaces (API) or protocol interfaces, presented and supported by HMS 22 for the client software applications. Interface layer 200 may be implemented with one or more web servers. In some examples, interface layer 200 may include communication circuitry.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

In some examples, data layer 204 includes an identifier/subscription service level (ID/SSL) database 258 including a plurality of identifiers each uniquely associated with an IMD or a patient. ID/SSL database 258 also includes an associated subscription service level with each of the plurality of identifiers. For example, ID/SSL database 258 may store identifier 76 (FIG. 2) and a subscription service level for patient 4. By storing identifier 76 and the subscription service level for patient 4 in ID/SSL database 258, HMS 22 may look up identifier 76 in ID/SSL database 258, determine a subscription service level for patient 4, generate one of more configuration messages including configuration data such as configuration data 198 (FIG. 3) or configuration data 78 (FIG. 2) which may be used by a device to define or facilitate functionality of IMD 10, any of computing devices 12, any of IoT devices 30, drone 46, and/or robot 48.

As shown in FIG. 4, each of services 230-242 is implemented in a modular form within HMS 22. Although shown as separate modules for each service, in some examples the functionality of two or more services may be combined into a single module or component. Each of services 230-242 may be implemented in software, hardware, or a combination of hardware and software. Moreover, services 230-242 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected an acute health event of patient and, in some examples, that the transmitting device confirmed or verified the detection. Event processor service 230 may initiate performance of any of the operations in response to detection of an acute health event ascribed herein to HMS 22, such as communicating with patient 4, bystander 26, and care providers 40, activating the verification device or system (e.g., any of IoT devices 30, computing device(s) 12, drone 46, or robot 48) and, in some cases, analyzing data to confirm or override the detection of the acute health event by IMD 10. In some examples, rather than actually verifying the acute health event, the verification device or system may transmit the sensed physiological parameters to HMS 22 and HMS 22 may verify the acute health event. In some examples, such features may be based on a subscription service level of patient 4.

Record management service 238 may store the patient data included in a received alert message within event records 252. Alert service 232 may package the some or all of the data from the event record, in some cases with additional information as described herein, into one more alert messages for transmission to bystander 26 and/or care providers 40. Care provider data 256 may store data used by alert service 232 to identify to whom to send alerts based on locations of potential bystanders 26 and care providers 40 relative to a location of patient 4 and/or applicability of the care provided by care providers 40 to the acute health event experienced by patient 4. In some examples, records management service 238 may, based on a subscription service level of patient 4, generate a report 248 including patient data for transmission to computing device 12A and/or 12B or any of IoT devices 30. Report 238 may include information regarding patient physiological parameters sensed by system 2. In some examples, report 238 may include a patient risk.

Patient risk analyzer 242 may determine a patient risk based on physiological parameters sensed by IMD 10 and/or other devices within system 2 (both of FIG. 1) that include sensors. Additionally, or alternatively, patient risk analyzer 244 may determine a patient risk based on EHR 24 (FIG. 1). In some examples, patient risk analyzer 242 may work with record management 238 to provide the patient risk assessment to record management 238 to be included in report 248. In some examples, determination of the patient risk may be based on a subscription service level of patient 4.

Patient risk analyzer 244 may generate an indication 246 of a recommended subscription service level to patient 4. Interface layer 200 may transmit indication 246 to system 2. For example, interface layer 200 may transmit indication 246 to computing device 12A, such as through a short message service (SMS) message, an email, a voicemail, or another indication. Computing device 12A may in turn display or play the indication to inform patient 4 of the recommended subscription service level. This may be performed based on a change in health status of patient 4, based on an acute health event of patient 4, upon a request of patient 4 for a recommended subscription service level, or periodically.

Configuration message generator 240 may be configured to generate a configuration message 244 which may include information for configuring at least one device of system 2, such as IMD 10, computing device 12A, computing device 12B, any of IoT devices 30, drone 46, or robot 48. Interface layer 200 may transmit configuration message 244 to any of the devices of system 2.

In examples in which HMS 22 performs an analysis to confirm, verify, or override the detection of the acute health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules 84 used by IMD 10. In such examples, rules configuration service 234 may be configured to develop and maintain rules 196 and rules 84. Rules configuration service 234 may be configured to modify these rules based on event feedback data 254 that indicates whether the detections and confirmations of acute health events by IMD 10, computing device 12, and/or HMS 22 were accurate. Event feedback data 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices.

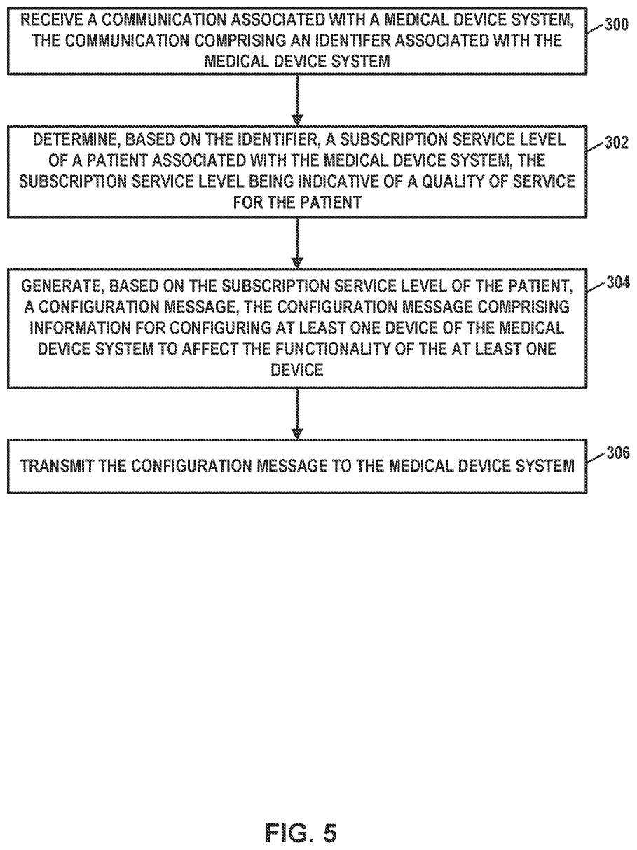

FIG. 5 is a flow diagram illustrating subscription service level techniques according to the present disclosure. Health monitoring system 22 may receive a communication associated with a medical device system, the communication comprising an identifier associated with the medical device system (300). For example, health monitoring system 22 may receive a communication from one of more of computing device 12A, computing device 12B, or IoT devices 30 including identifier 76 (FIG. 2) associated with IMD 10 or patient 4 of system 2 (FIG. 1).

Based on the identifier, health monitoring system 22 may determine a subscription service level of a patient associated with the medical device system, the subscription service level being indicative of a quality of service for the patient (302). For example, health monitoring system 22 may look up identifier 76 in ID/SSL database 258 to determine the subscription service level of patient 4.

Health monitoring system 22 may generate, based on the subscription service level of the patient, configuration message 242, configuration message 242 comprising information for configuring at least one device of the medical device system to affect the functionality of the at least one device (304). For example, configuration message generator 240 of health monitoring system 22 may generate configuration data 78 (FIG. 2) or configuration data 198 (FIG. 3) and may include configuration data 78 or configuration data 198 in configuration message 242.

Health monitoring system 22 may transmit the configuration message to the medical device system (306). For example, health monitoring system 22 may transmit the configuration message to IMD 10, to a computing device, such as computing device 12A, computing device 12B, or any of IoT devices 30. The configuration data may be used by IMD 10 and/or the computing device(s) to configure the functionality of IMD 10 and/or the computing device(s). For example, processing circuitry of IMD 10 or of a computing device may run a script or an application to effectuate functionality based on the configuration data in the configuration message.

In some examples, affecting the functionality comprises enabling connectivity between the at least one device of the medical device system to a larger number of devices than prior to the configuring. For example, the information of the configuration message may affect the functionality of the at least one device by at least enabling connectivity between the at least one device of the medical device system to a larger number of devices than prior to the configuring. In some examples, affecting the functionality comprises enabling prioritized communication between the at least one device of the medical device system and an emergency medical service, a drone, or a class of responders. For example, the information of the configuration message may affect the functionality of the at least one device by at least enabling prioritized communication between the at least one device of the medical device system and an emergency medical service, a drone, or a class of responders. In some examples, affecting the functionality comprises enabling the at least one device of the medical device system to communicate with at least one patient associated responder device. For example, the information of the configuration message may affect the functionality of the at least one device by at least enabling the at least one device of the medical device system to communicate with at least one patient associated responder device.

In some examples, affecting the functionality comprises facilitating a self-test of an implantable medical device of the medical device system. For example, the information of the configuration message may affect the functionality of the at least one device by at least facilitating a self-test of an implantable medical device of the medical device system. In some examples, affecting the functionality comprises configuring a mesh network comprising the at least one device of the medical device system. For example, the information of the configuration message may affect the functionality of the at least one device by at least configuring a mesh network comprising the at least one device of the medical device system. In some examples, the mesh network comprises a plurality of smart phones.

In some examples, health monitoring system 22 may determine a patient risk level of the patient. For example, heath monitoring system 22 may access EHR 24 (FIG. 1) and/or sensed physiological parameters of patient 4 and patient risk analyzer 244 (FIG. 4) may determine the patient risk level of patient 4. In some examples, health monitoring system 22 may determine a recommended subscription service level for the patient based on the patient risk level of the patient and health monitoring system 22 may transmit an indication 246 of the recommended subscription service level to the patient via the medical device system. For example, health monitoring system 22 may send an SMS message, an email, a voicemail, or another indication of the recommended subscription service level, to computing device 12A, computing device 12B, or any of IoT devices 30. In some examples, the subscription level is based at least in part on the patient risk level of patient 4. In some examples, health monitoring system 22 may transmit message 248 to the medical device system, message 246 comprising information regarding the patient risk level. In some examples, the information regarding the patient risk level comprises information regarding patient physiological parameters sensed by the medical device system.

In some examples, the subscription service level comprises a charge per instance level or a charge per continuous service level. In some examples, the at least one device includes at least one of an IMD, a smartphone, a desktop computer, a laptop computer, a table computer, a wearable device, an IoT device, a drone, or a robot. In some examples, the IMD includes an ICM.

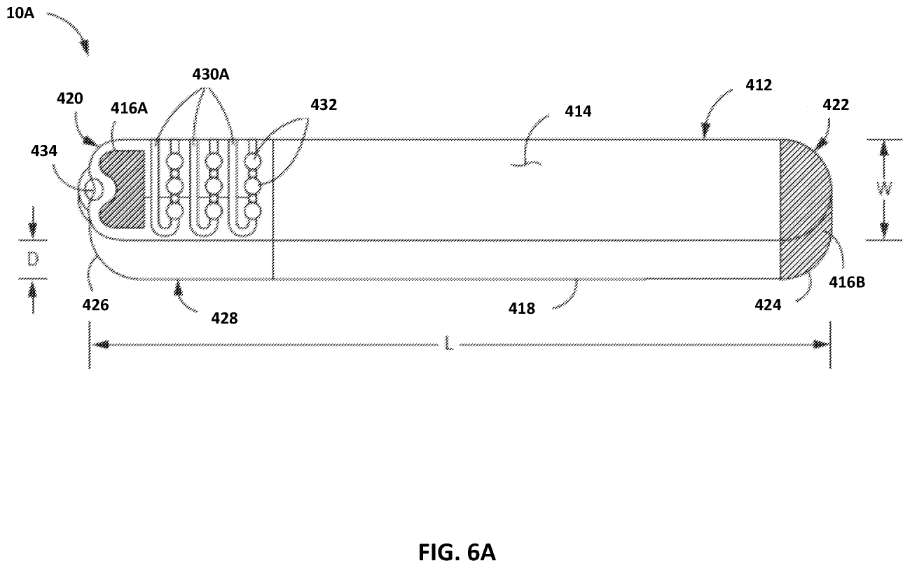

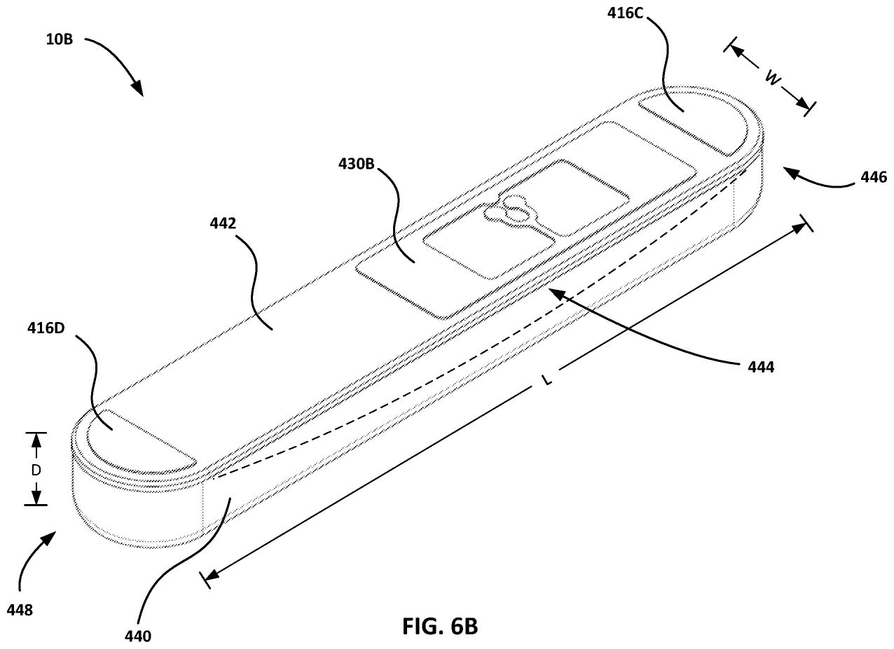

FIG. 6A is a perspective drawing illustrating an IMD 10A, which may be an example configuration of IMD 10 of FIGS. 1 and 2 as an ICM. In the example shown in FIG. 6A, IMD 10A may be embodied as a monitoring device having housing 412, proximal electrode 416A and distal electrode 416B. Housing 412 may further comprise first major surface 414, second major surface 418, proximal end 420, and distal end 422. Housing 412 encloses electronic circuitry located inside the IMD 10A and protects the circuitry contained therein from body fluids. Housing 412 may be hermetically sealed and configured for subcutaneous implantation. Electrical feedthroughs provide electrical connection of electrodes 416A and 416B.

In the example shown in FIG. 6A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 6A includes radial asymmetries (notably, the rectangular shape) along the longitudinal axis that maintains the device in the proper orientation following insertion. For example, the spacing between proximal electrode 416A and distal electrode 416B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 6A, once inserted within the patient, the first major surface 414 faces outward, toward the skin of the patient while the second major surface 418 is located opposite the first major surface 414. In addition, in the example shown in FIG. 6A, proximal end 420 and distal end 422 are rounded to reduce discomfort and irritation to surrounding tissue once inserted under the skin of the patient. IMD 10A, including instrument and method for inserting IMD 10 is described, for example, in U.S. Patent Publication No. 2014/0276928, incorporated herein by reference in its entirety.

Proximal electrode 416A is at or proximate to proximal end 420, and distal electrode 416B is at or proximate to distal end 422. Proximal electrode 416A and distal electrode 416B are used to sense cardiac EGM signals, e.g., ECG signals, thoracically outside the ribcage, which may be sub-muscularly or subcutaneously. Cardiac signals may be stored in a memory of IMD 10A, and data may be transmitted via integrated antenna 430A to another device, which may be another implantable device or an external device, such as external device 412. In some example, electrodes 416A and 416B may additionally or alternatively be used for sensing any bio-potential signal of interest, which may be, for example, an EGM, EEG, EMG, or a nerve signal, or for measuring impedance, from any implanted location.

In the example shown in FIG. 6A, proximal electrode 416A is at or in close proximity to the proximal end 420 and distal electrode 416B is at or in close proximity to distal end 422. In this example, distal electrode 416B is not limited to a flattened, outward facing surface, but may extend from first major surface 414 around rounded edges 424 and/or end surface 426 and onto the second major surface 418 so that the electrode 416B has a three-dimensional curved configuration. In some examples, electrode 416B is an uninsulated portion of a metallic, e.g., titanium, part of housing 412.

In the example shown in FIG. 6A, proximal electrode 416A is located on first major surface 414 and is substantially flat, and outward facing. However, in other examples proximal electrode 416A may utilize the three dimensional curved configuration of distal electrode 416B, providing a three dimensional proximal electrode (not shown in this example). Similarly, in other examples distal electrode 416B may utilize a substantially flat, outward facing electrode located on first major surface 414 similar to that shown with respect to proximal electrode 416A.

The various electrode configurations allow for configurations in which proximal electrode 416A and distal electrode 416B are located on both first major surface 414 and second major surface 418. In other configurations, such as that shown in FIG. 6A, only one of proximal electrode 416A and distal electrode 416B is located on both major surfaces 414 and 418, and in still other configurations both proximal electrode 416A and distal electrode 416B are located on one of the first major surface 414 or the second major surface 418 (e.g., proximal electrode 416A located on first major surface 414 while distal electrode 416B is located on second major surface 418). In another example, IMD 10A may include electrodes on both major surface 414 and 418 at or near the proximal and distal ends of the device, such that a total of four electrodes are included on IMD 10A. Electrodes 416A and 416B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 6A, proximal end 420 includes a header assembly 428 that includes one or more of proximal electrode 416A, integrated antenna 430A, anti-migration projections 432, and/or suture hole 434. Integrated antenna 430A is located on the same major surface (i.e., first major surface 414) as proximal electrode 416A and is also included as part of header assembly 428. Integrated antenna 430A allows IMD 10A to transmit and/or receive data. In other examples, integrated antenna 430A may be formed on the opposite major surface as proximal electrode 416A, or may be incorporated within the housing 412 of IMD 10A. In the example shown in FIG. 6A, anti-migration projections 432 are located adjacent to integrated antenna 430A and protrude away from first major surface 414 to prevent longitudinal movement of the device. In the example shown in FIG. 6A, anti-migration projections 432 include a plurality (e.g., nine) small bumps or protrusions extending away from first major surface 414. As discussed above, in other examples anti-migration projections 432 may be located on the opposite major surface as proximal electrode 416A and/or integrated antenna 430A. In addition, in the example shown in FIG. 6A, header assembly 428 includes suture hole 434, which provides another means of securing IMD 10A to the patient to prevent movement following insertion. In the example shown, suture hole 434 is located adjacent to proximal electrode 416A. In one example, header assembly 428 is a molded header assembly made from a polymeric or plastic material, which may be integrated or separable from the main portion of IMD 10A.

FIG. 6B is a perspective drawing illustrating another IMD 10B, which may be another example configuration of IMD 10 from FIGS. 1 and 2 as an ICM. IMD 10B of FIG. 6B may be configured substantially similarly to IMD 10A of FIG. 6A, with differences between them discussed herein.

IMD 10B may include a leadless, subcutaneously-implantable monitoring device, e.g. an ICM. IMD 10B includes housing having a base 440 and an insulative cover 442. Proximal electrode 416C and distal electrode 416D may be formed or placed on an outer surface of cover 442. Various circuitries and components of IMD 10B, e.g., described above with respect to FIG. 2, may be formed or placed on an inner surface of cover 442, or within base 440. In some examples, a battery or other power source of IMD 10B may be included within base 440. In the illustrated example, antenna 430B is formed or placed on the outer surface of cover 442, but may be formed or placed on the inner surface in some examples. In some examples, insulative cover 442 may be positioned over an open base 440 such that base 440 and cover 442 enclose the circuitries and other components and protect them from fluids such as body fluids. The housing including base 440 and insulative cover 442 may be hermetically sealed and configured for subcutaneous implantation.

Circuitries and components may be formed on the inner side of insulative cover 442, such as by using flip-chip technology. Insulative cover 442 may be flipped onto a base 440. When flipped and placed onto base 440, the components of IMD 10B formed on the inner side of insulative cover 442 may be positioned in a gap 444 defined by base 440. Electrodes 416C and 416D and antenna 430B may be electrically connected to circuitry formed on the inner side of insulative cover 442 through one or more vias (not shown) formed through insulative cover 442. Insulative cover 442 may be formed of sapphire (i.e., corundum), glass, parylene, and/or any other suitable insulating material. Base 440 may be formed from titanium or any other suitable material (e.g., a biocompatible material). Electrodes 416C and 416D may be formed from any of stainless steel, titanium, platinum, iridium, or alloys thereof. In addition, electrodes 416C and 416D may be coated with a material such as titanium nitride or fractal titanium nitride, although other suitable materials and coatings for such electrodes may be used.

In the example shown in FIG. 6B, the housing of IMD 10B defines a length L, a width W and thickness or depth D and is in the form of an elongated rectangular prism wherein the length L is much larger than the width W, which in turn is larger than the depth D, similar to IMD 10A of FIG. 6A. For example, the spacing between proximal electrode 416C and distal electrode 416D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 6B, once inserted subcutaneously within the patient, outer surface of cover 442 faces outward, toward the skin of the patient. In addition, as shown in FIG. 6B, proximal end 446 and distal end 448 are rounded to reduce discomfort and irritation to surrounding tissue once inserted under the skin of the patient. In addition, edges of IMD 10B may be rounded.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

This disclosure includes the following non-limiting examples.

Example 1A. A system comprising: communication circuitry configured to communicate with a medical device system; memory communicatively coupled to the communication circuitry and being configured to store an identifier associated with the medical device system and a subscription service level of a patient; and processing circuitry communicatively coupled to the communication circuitry and the memory, the processing circuitry being configured to: control the communication circuitry to receive a communication associated with the medical device system, the communication comprising an identifier associated with the medical device system; determine, based on the identifier, the subscription service level of the patient, the subscription service level of the patient being indicative of a quality of service for the patient; generate, based on the subscription service level of the patient, a configuration message, the configuration message comprising information for configuring at least one device of the medical device system to affect the functionality of the at least one device; and control the communication circuitry to transmit the configuration message to the medical device system.

Example 2A. The system of example 1A, wherein affecting the functionality comprises enabling connectivity between the at least one device of the medical device system to a larger number of devices than prior to the configuring.

Example 3A. The system of example 1A or example 2A, wherein affecting the functionality comprises enabling prioritized communication between the at least one device of the medical device system and an emergency medical service, a drone, or a class of responders.

Example 4A. The system of any one or more of examples 1A-3A, wherein affecting the functionality comprises enabling the at least one device of the medical device system to communicate with at least one patient associated responder device.

Example 5A. The system of any one or more of examples 1A-4A, wherein affecting the functionality comprises facilitating a self-test of an implantable medical device of the medical device system.

Example 6A. The system of any one or more of examples 1A-5A, wherein affecting the functionality comprises configuring a mesh network comprising the at least one device of the medical device system.

Example 7A. The system of example 6A, wherein the mesh network comprises a plurality of smart phones.

Example 8A. The system of any one or more of examples 1A-7A, wherein the processing circuitry is further configured to: determine a patient risk level of the patient.

Example 9A. The system of example 8A, wherein the processing circuitry is further configured to: determine a recommended subscription service level for the patient based on the patient risk level of the patient; and control the communication circuitry to transmit an indication of the recommended the subscription level to the patient via the medical device system.

Example 10A. The system of example 8A or example 9A, wherein the subscription level is based at least in part on the patient risk level of the patient.

Example 11A. The system of any one or more of examples 8A-10A, wherein the processing circuitry is further configured to: control the communication circuitry to transmit a message to the medical device system, the message comprising information regarding the patient risk level.

Example 12A. The system of example 11A, wherein the information regarding the patient risk level comprises information regarding patient physiological parameters sensed by the medical device system.

Example 13A. The system of any one or more of examples 1A-12A, wherein the subscription service level comprises a charge per instance level or a charge per continuous service level.

Example 14A. A method comprising: receiving, by communication circuitry, a communication associated with a medical device system, the communication comprising an identifier associated with the medical device system; determining, by processing circuitry and based on the identifier, a subscription service level of a patient associated with the medical device system, the subscription service level being indicative of a quality of service for the patient; generating, by the processing circuitry and based on the subscription service level of the patient, a configuration message, the configuration message comprising information for configuring at least one device of the medical device system to affect the functionality of the at least one device; and transmitting, by the communication circuitry, the configuration message to the medical device system.

Example 15A. The method of example 14A, wherein affecting the functionality comprises enabling connectivity between the at least one device of the medical device system to a larger number of devices than prior to the configuring.

Example 16A. The method of example 14A or example 15A, wherein affecting the functionality comprises enabling prioritized communication between the at least one device of the medical device system and an emergency medical service, a drone, or a class of responders.

Example 17A. The method of any one or more of examples 14A-16A, wherein affecting the functionality comprises enabling the at least one device of the medical device system to communicate with at least one patient associated responder device.

Example 18A. The method of any one or more of examples 14A-17A, wherein affecting the functionality comprises facilitating a self-test of an implantable medical device of the medical device system.

Example 19A. The method of any one or more of examples 14A-18A, wherein affecting the functionality comprises configuring a mesh network comprising the at least one device of the medical device system.

Example 20A. The method of example 19A, wherein the mesh network comprises a plurality of smart phones.

Example 21A. The method of any one or more of examples 14A-20A, further comprising: determining, by the processing circuitry, a patient risk level of the patient.

Example 22A. The method of example 21A, further comprising: determining, by the processing circuitry, a recommended subscription service level for the patient based on the patient risk level of the patient; and transmitting, by the communication circuitry, an indication of the recommended the subscription level to the patient via the medical device system.

Example 23A. The method of example 21A or example 22A, wherein the subscription level is based at least in part on the patient risk level of the patient.

Example 24A. The method of any one or more of examples 21A-23A, further comprising: transmitting, by the communication circuitry, a message to the medical device system, the message comprising information regarding the patient risk level.

Example 25A. The method of example 24A, wherein the information regarding the patient risk level comprises information regarding patient physiological parameters sensed by the medical device system.

Example 26A. The method of any one or more of examples 14A-25A, wherein the subscription service level comprises a charge per instance level or a charge per continuous service level.

Example 27A. A non-transitory computer-readable storage medium storing instructions that, when executed, cause processing circuitry to: control communication circuitry to receive a communication associated with a medical device system, the communication comprising an identifier associated with the medical device system; based on the identifier, determine a subscription service level of a patient associated with the medical device system, the subscription service level being indicative of a quality of service for the patient; based on the subscription service level of the patient, generate a configuration message, the configuration message comprising information for configuring at least one device of the medical device system to affect the functionality of the at least one device; and control the communication circuitry to transmit the configuration message to the medical device system.

Example 1B. A computing system comprising: communication circuitry configured to communicate with one or more devices of a medical device system, the medical device system comprising at least an implantable medical device (IMD); memory communicatively coupled to the communication circuitry and being configured to store an identifier associated with the medical device system and a subscription service level of a patient; and processing circuitry communicatively coupled to the communication circuitry and the memory, the processing circuitry being configured to: control the communication circuitry to receive a communication associated with the medical device system, the communication comprising an identifier associated with the medical device system; determine, based on the identifier, the subscription service level of the patient, the subscription service level of the patient being indicative of a quality of service for the patient; generate, based on the subscription service level of the patient, a configuration message, the configuration message comprising information for configuring at least one device of the medical device system to affect the functionality of the at least one device; and control the communication circuitry to transmit the configuration message to the medical device system.

Example 2B. The system of example 1B, wherein the information of the configuration message affects the functionality of the at least one device by at least enabling connectivity between the at least one device of the medical device system to a larger number of devices than prior to the configuring.

Example 3B. The system of example 1B or example 2B, wherein the information of the configuration message affects the functionality of the at least one device by at least enabling prioritized communication between the at least one device of the medical device system and an emergency medical service, a drone, or a class of responders.

Example 4B. The system of any one or more of examples 1B-3B, wherein the information of the configuration message affects the functionality of the at least one device by at least enabling the at least one device of the medical device system to communicate with at least one patient associated responder device.

Example 5B. The system of any one or more of examples 1B-4B, wherein the information of the configuration message affects the functionality of the at least one device by at least facilitating a self-test of an implantable medical device of the medical device system.

Example 6B. The system of any one or more of examples 1B-5B, wherein the information of the configuration message affects the functionality of the at least one device by at least configuring a mesh network comprising the at least one device of the medical device system.

Example 7B. The system of example 6B, wherein the mesh network comprises a plurality of smart phones.

Example 8B. The system of any one or more of examples 1B-7B, wherein the processing circuitry is further configured to: determine a patient risk level of the patient.

Example 9B. The system of example 8B, wherein the processing circuitry is further configured to: determine a recommended subscription service level for the patient based on the patient risk level of the patient; and control the communication circuitry to transmit an indication of the recommended the subscription level to the patient via the medical device system.

Example 10B. The system of example 8B or example 9B, wherein the subscription level is based at least in part on the patient risk level of the patient.

Example 11B. The system of any one or more of examples 8B-10B, wherein the processing circuitry is further configured to: control the communication circuitry to transmit a message to the medical device system, the message comprising information regarding the patient risk level.

Example 12B. The system of example 11B, wherein the information regarding the patient risk level comprises information regarding patient physiological parameters sensed by the medical device system.

Example 13B. The system of any one or more of examples 1B-12B, wherein the subscription service level comprises a charge per instance level or a charge per continuous service level.

Example 14B. The system of any one or more of examples 1B-13B, wherein the at least one device comprises at least one of the IMD, a smartphone, a desktop computer, a laptop computer, a table computer, a wearable device, an IoT device, a drone, or a robot.

Example 15B. The system of any one or more of examples 1B-14B, wherein the IMD comprises an insertable cardiac monitor (ICM).

Various examples have been described. These and other examples are within the scope of the following claims.

1. A computing system comprising:
communication circuitry configured to communicate with one or more devices of a medical device system, the medical device system comprising at least an implantable medical device (IMD);
memory communicatively coupled to the communication circuitry and being configured to store an identifier associated with the medical device system and a subscription service level of a patient; and
processing circuitry communicatively coupled to the communication circuitry and the memory, the processing circuitry being configured to:
control the communication circuitry to receive a communication associated with the medical device system, the communication comprising an identifier associated with the medical device system;
determine, based on the identifier, the subscription service level of the patient, the subscription service level of the patient being indicative of a quality of service for the patient;
generate, based on the subscription service level of the patient, a configuration message, the configuration message comprising information for configuring at least one device of the medical device system to affect the functionality of the at least one device, wherein the information of the configuration message affects the functionality of the at least one device by at least enabling connectivity between the at least one device of the medical device system to a larger number of devices than prior to configuring; and
control the communication circuitry to transmit the configuration message to the medical device system.

2-15. (canceled)

16. The system of claim 1, wherein the information of the configuration message affects the functionality of the at least one device by at least enabling prioritized communication between the at least one device of the medical device system and an emergency medical service, a drone, or a class of responders.

17. The system of claim 1, wherein the information of the configuration message affects the functionality of the at least one device by at least enabling the at least one device of the medical device system to communicate with at least one patient associated responder device.

18. The system of claim 1, wherein the information of the configuration message affects the functionality of the at least one device by at least facilitating a self-test of the implantable medical device of the medical device system.

19. The system of claim 1, wherein the information of the configuration message affects the functionality of the at least one device by at least configuring a mesh network comprising the at least one device of the medical device system.

20. The system of claim 19, wherein the mesh network comprises a plurality of smart phones.

21. The system of claim 1, wherein the processing circuitry is further configured to:
determine a patient risk level of the patient.

22. The system of claim 21, wherein the processing circuitry is further configured to:
determine a recommended subscription service level for the patient based on the patient risk level of the patient; and
control the communication circuitry to transmit an indication of the recommended the subscription level to the patient via the medical device system.

23. The system of claim 21, wherein the subscription level is based at least in part on the patient risk level of the patient.

24. The system of claim 21, wherein the processing circuitry is further configured to:
control the communication circuitry to transmit a message to the medical device system, the message comprising information regarding the patient risk level.

25. The system of claim 24, wherein the information regarding the patient risk level comprises information regarding patient physiological parameters sensed by the medical device system.

26. The system of claim 1, wherein the subscription service level comprises a charge per instance level or a charge per continuous service level.

27. The system of claim 1, wherein the at least one device comprises at least one of the IMD, a smartphone, a desktop computer, a laptop computer, a table computer, a wearable device, an IoT device, a drone, or a robot.

28. The system of claim 1, wherein the IMD comprises an insertable cardiac monitor (ICM).

29. A method comprising:
controlling, by processing circuitry of a computing device, communication circuitry of the computing device configured to communicate with one or more devices of a medical device system comprising at least an implantable medical device to receive a communication associated with the medical device system, the communication comprising an identifier associated with the medical device system;
determining, by the processing circuitry and based on the identifier, a subscription service level of a patient, the subscription service level of the patient being indicative of a quality of service for the patient;
generating, by the processing circuitry and based on the subscription service level of the patient, a configuration message, the configuration message comprising information for configuring at least one device of the medical device system to affect the functionality of the at least one device, wherein the information of the configuration message affects the functionality of the at least one device by at least enabling connectivity between the at least one device of the medical device system to a larger number of devices than prior to configuring; and
controlling, by the processing circuitry, the communication circuitry to transmit the configuration message to the medical device system.

30. The method of claim 29, wherein the information of the configuration message affects the functionality of the at least one device by at least enabling prioritized communication between the at least one device of the medical device system and an emergency medical service, a drone, or a class of responders.

31. The method of claim 29, wherein the information of the configuration message affects the functionality of the at least one device by at least enabling the at least one device of the medical device system to communicate with at least one patient associated responder device.

32. The method of claim 29, wherein the information of the configuration message affects the functionality of the at least one device by at least facilitating a self-test of the implantable medical device of the medical device system.

33. The method of claim 29, wherein the information of the configuration message affects the functionality of the at least one device by at least configuring a mesh network comprising the at least one device of the medical device system.

34. A non-transitory computer-readable medium storing instructions that when executed cause processing circuitry to:
control communication circuitry of a computing device configured to communicate with one or more device of a medical device system comprising at least an implantable medical device to receive a communication associated with the medical device system, the communication comprising an identifier associated with the medical device system;
determine, based on the identifier, the subscription service level of a patient, the subscription service level of the patient being indicative of a quality of service for the patient;
generate, based on the subscription service level of the patient, a configuration message, the configuration message comprising information for configuring at least one device of the medical device system to affect the functionality of the at least one device, wherein the information of the configuration message affects the functionality of the at least one device by at least enabling connectivity between the at least one device of the medical device system to a larger number of devices than prior to configuring; and
control the communication circuitry to transmit the configuration message to the medical device system.
